(19) United States
(12) Patent Application Publication
(10) Pub. No.: US 2012/0154748 A1
(43) Pub. Date: Jun. 21, 2012

(54) FUNDUS CAMERA

(75) Inventors: Hiroyuki Inoue, Kawasaki-shi (JP); Tomoyuki Iwanaga, Yokohama-shi (JP); Shinya Tanaka, Katowice (PL)

(73) Assignee: CANON KABUSHIKI KAISHA, Tokyo (JP)

(21) Appl. No.: 13/393,001

(22) PCT Filed: Aug. 30, 2010
(86) PCT No.: PCT/JP2010/005332

§ 371 Date: Feb. 28, 2012

(30) Foreign Application Priority Data
Sep. 1, 2009 (JP) 2009-201290

(51) Int. Cl. A61B 3/14

(52) U.S. Cl. 351/206

(57) ABSTRACT

If a position of a focus lens is greatly displaced from a focus state when automatic focusing is started, and pattern recognition cannot be performed, the process proceeds to step S3. In step S3, the lens is sequentially driven until pattern recognition can be performed. If it is determined that the pattern recognition can be performed in step S2, a focus detection range is determined in S4. In step S5, an AF evaluation value of the range is calculated, and the value is stored in step S6.

### TECHNICAL FIELD

The present invention relates to a fundus camera used in an ophthalmic hospital or a group medical examination to photograph a fundus of an eye to be examined.

### BACKGROUND ART

When a fundus camera is to be focused on the fundus of the eye to be examined, an index is projected on the fundus. An index image is then observed via a focus lens of an observation photographing system, and the fundus camera is focused on the fundus based on the observed position of the index image.

Japanese Patent Application Laid-Open No. 5-95907 discusses a fundus camera that picks up a focus split index image which is split into two and projected on the fundus. The fundus camera then detects a focus state from each position of the focus split index image and attenuates the brightness of the index.

Further, Japanese Patent Application Laid-Open No. 8-275921 discusses an ophthalmic apparatus that projects a focus index on the fundus. The apparatus then picks up the focus index image using an imaging optical system and detects the focus state.

Furthermore, Japanese Patent Application Laid-Open No. 1-178237 discusses a modified example of an apparatus that electronically picks up an image during observation and performs automatic focusing (AF) by performing contrast detection of the picked-up image itself. More specifically, the apparatus focuses on a first range and a second range of the fundus using a high-frequency component of the fundus image and acquires distances from each range to a focus lens position in a direction of the optical axis.

However, a conventional fundus camera separates an area in which a fundus illumination light flux or a focus split index light flux is emitted, from an area in which an observation photographing light flux is emitted near a pupil of the eye to be examined. This is to eliminate reflected light by a cornea of the eye to be examined. If there is a difference among individuals in the aberration of an optical system of the eye to be examined, a focusing error may then be generated depending on the eye to be examined. More specifically, the error is generated when the fundus is photographed by only setting the focus split index position to a predetermined position, and an unfocused fundus image may thus be acquired.

To solve such a problem, there is an apparatus which electronically picks up an image during observation and performs automatic focusing by performing contrast detection of the picked-up image itself.

Such an apparatus solves the above-described problem in which the unfocused fundus image is acquired due to the focusing error depending on the eye to be examined. However, since a focus detection region is fixedly arranged with respect to a portion of the image pickup system, the following problem arises.

The distance in the depth direction of the fundus image is different depending on the region of the fundus. In a conventional AF detection in which a focus detection range is fixed, it thus becomes necessary to direct a line-of-sight of the eye to be examined so that the region to be focused matches the focus detection range.

Further, it becomes necessary to manually move the detection range when the focus detection range is movable, as in a general AF single-lens reflex camera. Furthermore, the AF detection position may change due to a movement of the eye to be examined.

### CITATION LIST

### Patent Literature

### [PTL 1]

### Japanese Patent Application Laid-Open No. 5-95907

### [PTL 2]

### Japanese Patent Application Laid-Open No. 8-275921

### [PTL 3]

### Japanese Patent Application Laid-Open No. 1-178237

### Summary Of Invention

The present invention is directed to a fundus camera that solves the above-described problems and is capable of easily performing alignment.

According to an aspect of the present invention, a fundus camera includes a fundus illumination optical system configured to illuminate a fundus of an eye to be examined, a fundus imaging optical system having a focus lens which is driven to focus on a fundus, a focus lens driving unit configured to drive the focus lens, a fundus image pickup unit arranged at a position which is conjugate to a fundus with respect to the fundus imaging optical system, a display monitor configured to display a fundus image acquired by the fundus image pickup unit, a focus state detection unit configured to detect an AF evaluation value indicating a level of a focus state based on an output signal from the fundus image pickup unit, and a lens drive control unit configured to drive the focus lens based on the AF evaluation value detected by the focus state detection unit, wherein the focus state detection unit includes a fundus position detection unit configured to detect with respect to an output from the fundus image pickup unit a specific region of a fundus image using a region pattern unique to a fundus region, and a focus detection range determination unit configured to determine a focus detection range based on an output from the fundus position detection unit, and calculates the AF evaluation value of the focus detection range determined by the focus detection range determination unit.

Further features and aspects of the present invention will become apparent from the following detailed description of exemplary embodiments with reference to the attached drawings.

### BRIEF DESCRIPTION OF DRAWINGS

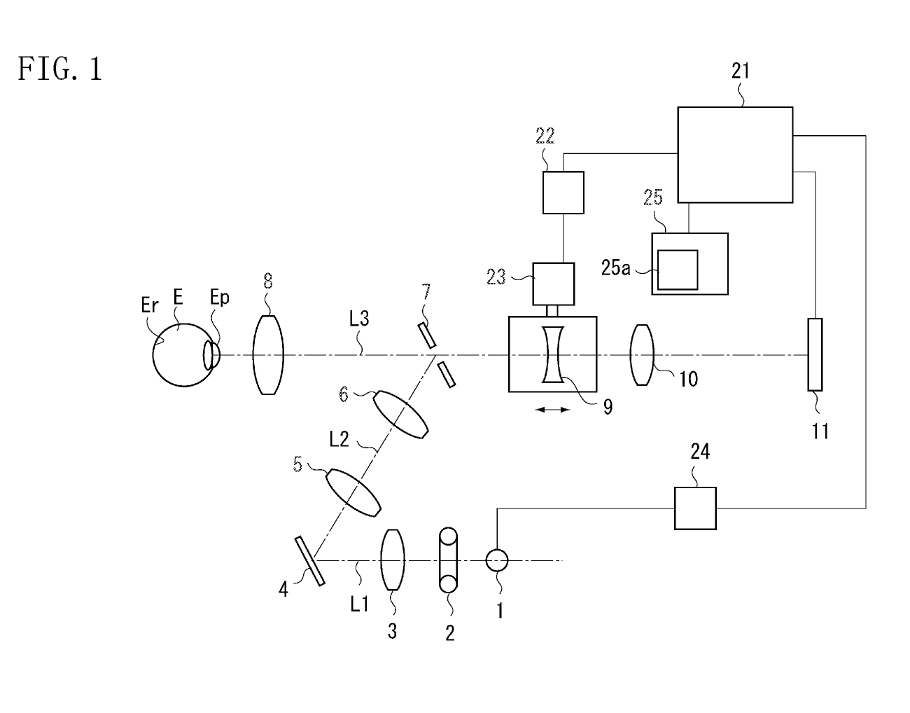

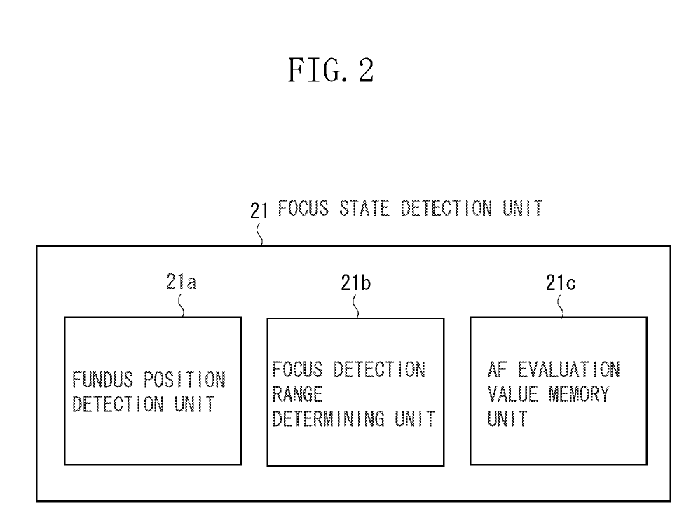

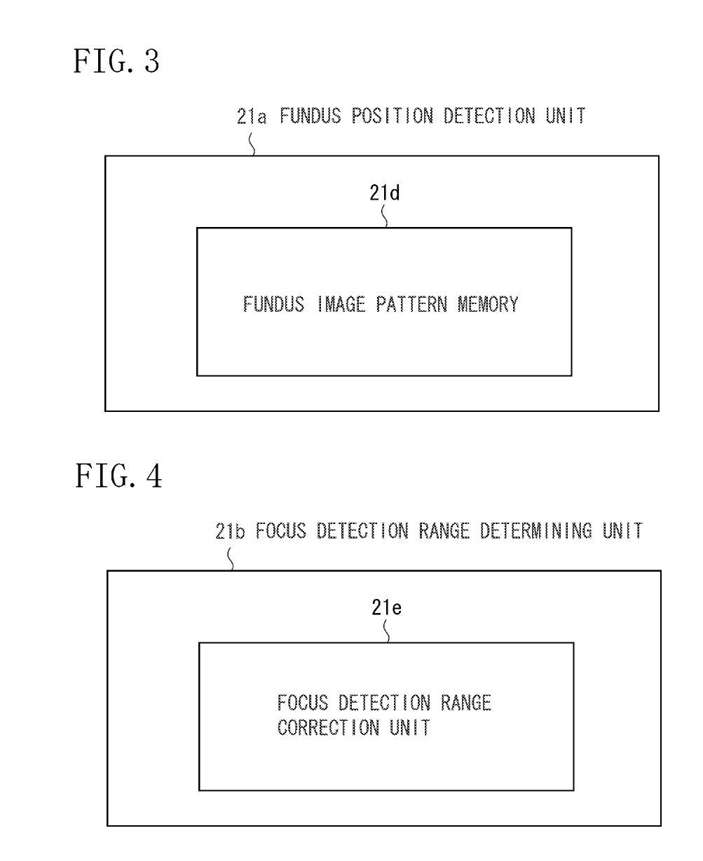

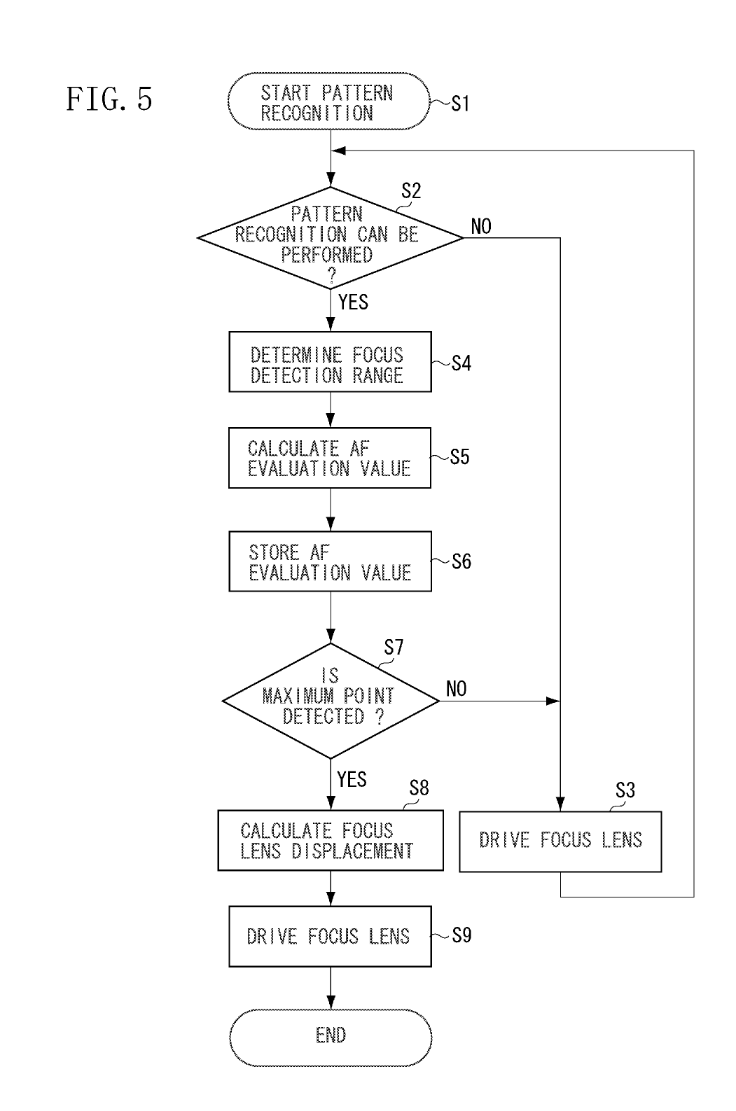

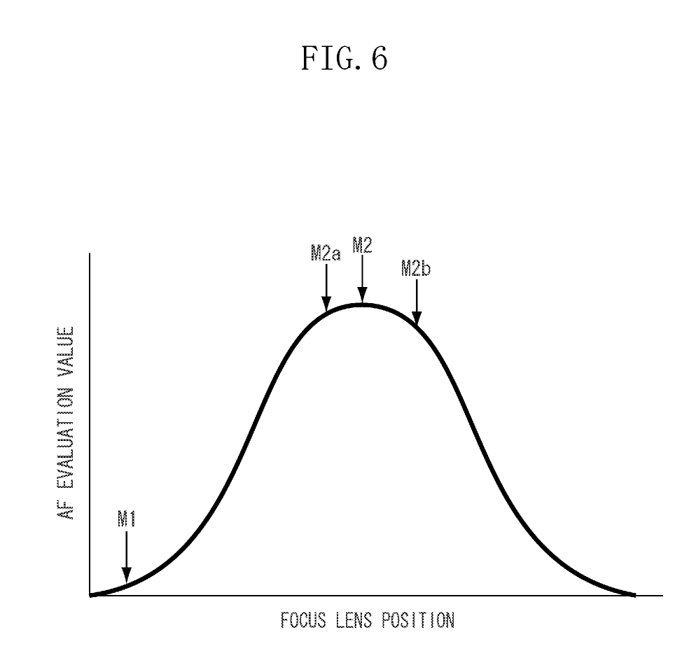

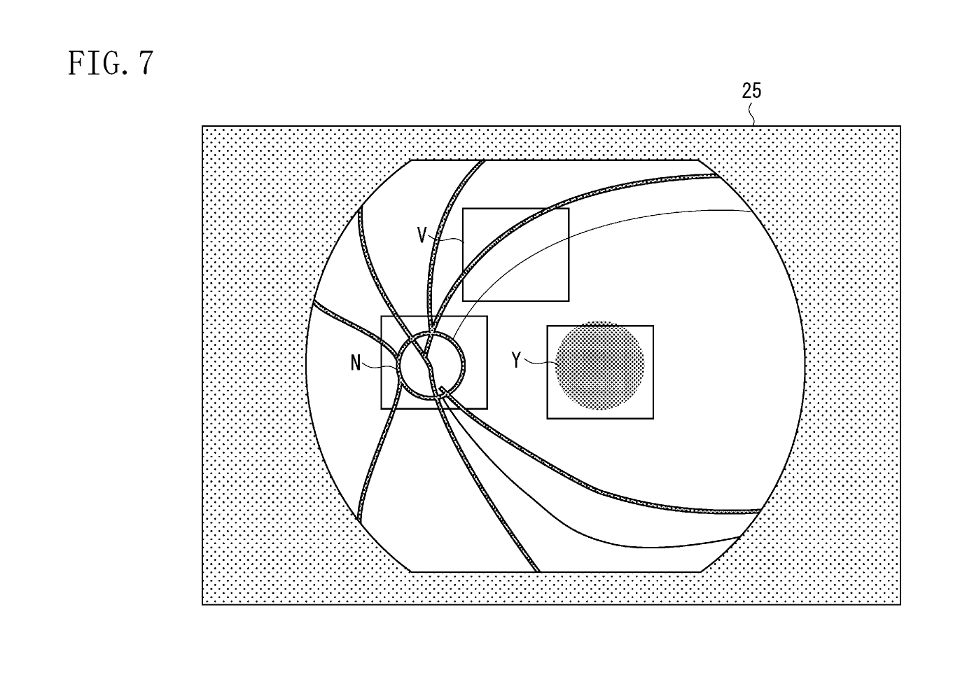

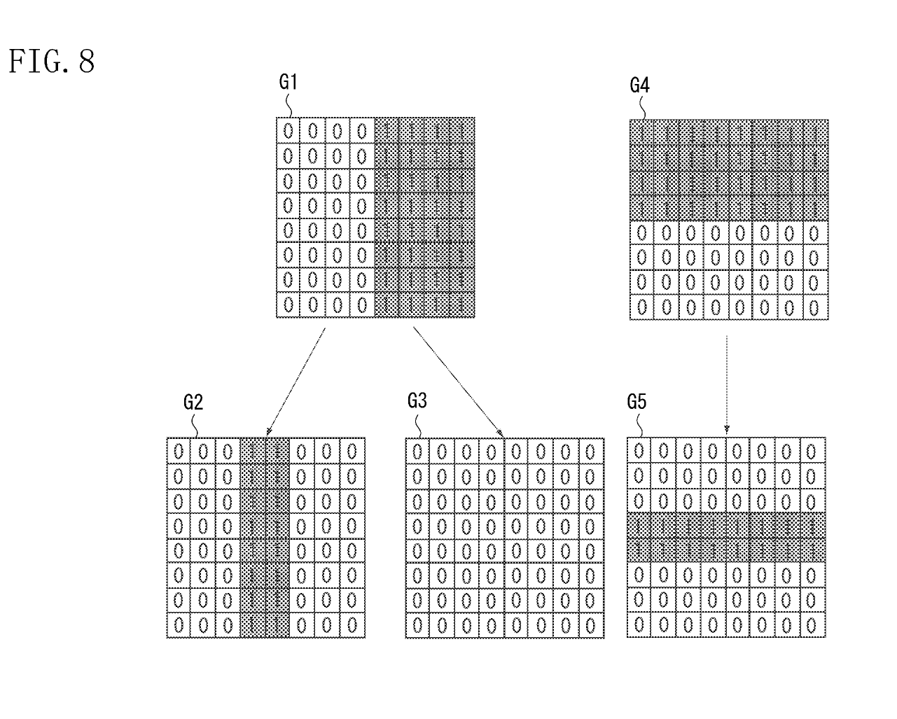

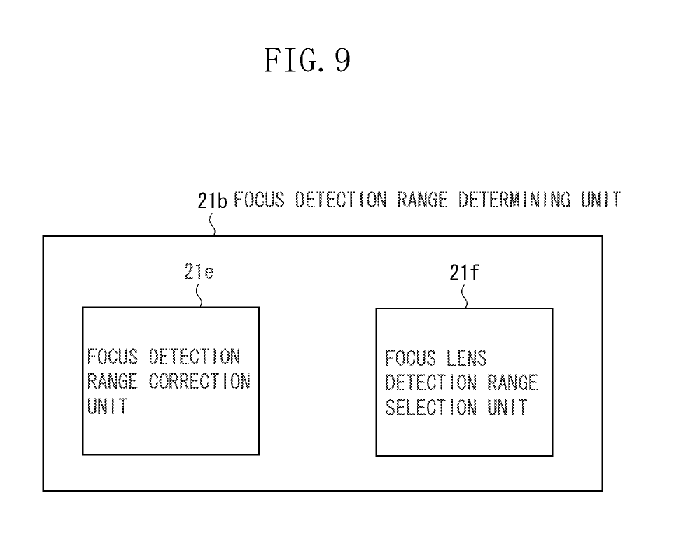

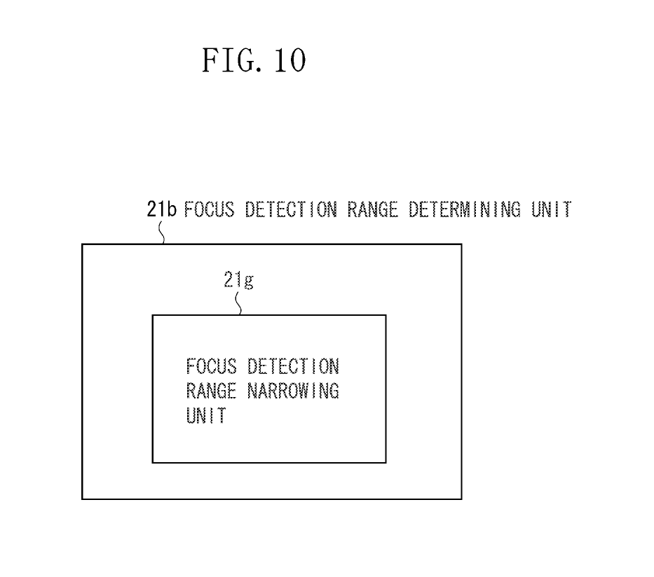

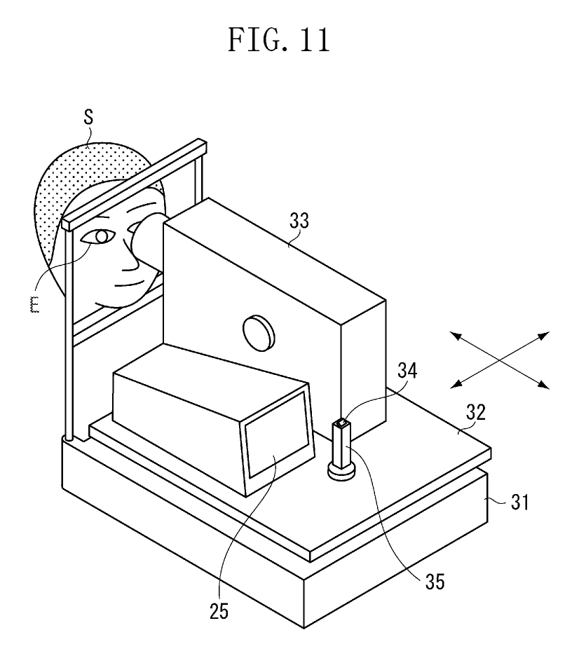

The accompanying drawings, which are incorporated in and constitute a part of the specification, illustrate exemplary embodiments, features, and aspects of the invention and, together with the description, serve to explain the principles of the invention.

[FIG. 1] FIG. 1 illustrates a configuration of a fundus camera according to a first exemplary embodiment of the present invention.

[FIG. 2] FIG. 2 illustrates a configuration of a focus state detection unit.

[FIG. 3] FIG. 3 illustrates a configuration of a fundus position detection unit.

[FIG. 4] FIG. 4 illustrates a configuration of a focus detection range determination unit.

[FIG. 5] FIG. 5 is a flowchart illustrating a control method.

[FIG. 6] FIG. 6 illustrates a basic principle of contrast detection.

[FIG. 7] FIG. 7 illustrates a fundus image displayed on a display monitor.

[FIG. 8] FIG. 8 illustrates a method for calculating an AF evaluation value.

[FIG. 9] FIG. 9 illustrates a configuration of a focus detection range determination unit according to a third exemplary embodiment of the present invention. [FIG. 10] FIG. 10 illustrates a configuration of a focus detection range determination unit according to a fourth exemplary embodiment of the present invention.

[FIG. 11] FIG. 11 illustrates an external view of a fundus camera.

[FIG. 12] FIG. 12 illustrates a configuration of a right/left eye detection unit.

### DESCRIPTION OF EMBODIMENTS

Various exemplary embodiments, features, and aspects of the invention will be described in detail below with reference to the drawings.

The first exemplary embodiment according to the present invention will be described below. FIG. 1 illustrates a configuration of the fundus camera. Referring to FIG. 1, a fundus illumination optical system is formed as described below. An observation light source 1, a photographing light source 2, a lens 3, and a mirror 4 are arranged on an optical axis L1. Relay lenses 5 and 6 and a perforated mirror 7 having an opening at the center are sequentially arranged on an optical axis L2 in a reflection direction of the mirror 4. An objective lens 8 is arranged opposite to an eye to be examined E on an optical axis L3 in the reflection direction of the perforated mirror 7. The observation light source 1 for illuminating the fundus is formed of a halogen lamp that emits ambient light, and the photographing light source 2 is formed of a stroboscopic tube that emits visible light.

A fundus imaging optical system in the fundus camera illustrated in FIG. 1 is configured as described below. A focus lens 9 for adjusting the focus by moving along the optical axis, a photographing lens 10, and a fundus image pickup unit 11 disposed at a position conjugate to a fundus Er are sequentially arranged at the rear of the perforated mirror 7 on the optical axis L3.

An output from the fundus image pickup unit 11 is transmitted to a focus state detection unit 21. Further, an output from the focus state detection unit 21 is transmitted to the focus lens 9 via a lens drive control unit 22 and a focus lens driving unit 23, also to the observation light source 1 via an illumination light quantity control unit 24, and is connected to a display monitor 25. A focus detection range display unit 25a is included in the display monitor 25.

An examiner observes the fundus image displayed on the display monitor 25 and adjusts using the observation light source 1 the alignment of the eye to be examined E with a chassis that includes the optical system. The examiner then adjusts the focus and photographs the fundus using the photographing light source 2.

The fundus camera according to the present exemplary embodiment includes an AF function that automatically adjusts the focus. The fundus camera is capable of displaying the focus detection range to the examiner by superposing a frame portion of the focus detection range display unit 25a on the fundus image acquired by the fundus image pickup unit 11. As a result, the fundus camera is capable of visually displaying the focus detection position to the user and thus improves the AF operability.

Focus detection in such a fundus camera is performed using contrast detection of the fundus image that is formed by the photographing light flux. The fundus camera is thus different from a conventional apparatus which projects the focus index via an anterior eye region outside the image pickup light flux. The fundus camera is capable of performing automatic focusing independent of the aberration of the optical system of the eye to be examined.

Referring to FIG. 2, the focus state detection unit 21 includes a fundus position detection unit 21a that detects a specific position of the fundus Er. The focus state detection unit 21 also includes a focus detection range determination unit 21b that determines the focus detection range based on a signal received from the fundus position detection unit 21a. Further, the focus state detection unit 21 includes an AF evaluation value storing unit 21c that stores the AF evaluation value and the position of the focus lens 9 when the AF evaluation value is acquired.

Referring to FIG. 3, the fundus position detection unit 21a includes a fundus image pattern memory 21d that stores region patterns which are reference images of the specific regions in the fundus image. The region pattern is used to extract the specific region from the fundus image. Position information of the specific region is acquired by performing pattern matching between the region pattern recorded in the fundus image pattern memory 21d and an output signal from the fundus image pickup unit 11. Further, the focus detection range determination unit 21b determines the range to be focused based on the fundus image specific region extracted by the fundus position detection unit 21a. However, it is desirable that the focus detection range determination unit 21b includes a focus detection range correction unit 21e as illustrated in FIG. 4, so that the examiner can correct the size of the focus detection range. The examiner uses the focus detection range correction unit 21e by operating a cursor with respect to the image on the display monitor 25.

The focus state detection unit 21 calculates the AF evaluation value of the focus detection range determined by the focus detection range determination unit 21b. The focus state detection unit 21 also stores information about the position of the focus lens 9 at that time in the AF evaluation value storing unit 21c.

FIG. 5 is a flowchart illustrating an AF control method. The examiner instructs start of the AF operation via an AF start switch (not illustrated). In step S1, the fundus camera then starts performing pattern recognition of the fundus image. In step S2, the fundus position detection unit 21a calculates a correlation function between the output from the fundus image pickup unit 11 and the region pattern of the fundus image specific region stored in the fundus image pattern memory 21d. The fundus position detection unit 21a then performs a comparison, and the ranges in which the calculated value becomes greater than or equal to a threshold value are determined to be the same range. The fundus position detection unit 21a thus determines whether pattern recognition can be performed.

If the position of the focus lens 9 is greatly displaced from the focused position when automatic focusing is started so that pattern recognition cannot be performed (NO in step S2), the process proceeds to step S3. In step S3, the fundus camera sequentially drives the focus lens 9 until pattern recognition can be performed . The pattern recognition is performed each time.

If pattern recognition can be performed (YES in step S2), the process proceeds to step S4. In step S4, the focus detection range determination unit 21b determines the focus detection range based on the output from the fundus position detection unit 21a. In step S5, the focus state detection unit 21 then calculates the AF evaluation value indicating a focus level of the focus detection range. The method for calculating the AF evaluation value will be described below. In step S6, the AF evaluation storing unit 21c stores the calculated AF evaluation value.

FIG. 6 illustrates a principle of the focus detection using contrast detection. Such a focus detection method is performed based on a specific high-frequency component of a luminance signal, which obtains a maximum value when focused. The focus state detection unit 21 thus detects and uses as the AF evaluation value the high-frequency component of the input luminance signal. Referring to FIG. 6, a position of the focus lens is indicated on a horizontal axis, and the AF evaluation value is indicated on a vertical axis. The AF evaluation value becomes the maximum value at a focus position M2 and decreases at a position M1 which is greatly out of focus. According to the present exemplary embodiment, focus correction which matches the aberration of the optical system of the human eye is performed using the contrast detection principle.

In step S7, the fundus camera determines whether the maximum point, i.e., the position M2 illustrated in FIG. 6, is included in the AF evaluation values stored in step S6, using the principle of contrast detection. Since the determination of the maximum point cannot be performed in an initial determination performed in step S7, the process proceeds to step S3. In step S3, the fundus camera drives the focus lens 9.

If the maximum point is detected in the AF evaluation values (YES in step S7), the process proceeds to step S8. In step S8, the focus state detection unit 21 calculates a displacement of the focus lens 9. The displacement of the focus lens 9 in step S8 is an amount the focus lens 9 is driven to the position at which the maximum point M2 of the AF evaluation value is detected. In step S9, the lens drive control unit 22 transmits a signal to the focus lens driving unit 23 based on the focus lens displacement calculated in step S8 and drives the focus lens 9. Automatic focusing thus ends.

In the above-described process, the focus lens 9 is driven and the automatic focusing ends in step S9 based on the displacement of the focus lens 9 calculated in step S8. However, the processes of step S2 to step S5 maybe performed after performing step S9 to calculate the AF evaluation value. The calculated AF value is then compared with the AF evaluation value in which the maximum point is first determined. Automatic focusing may thus end when the difference between the AF evaluation values become less than or equal to a threshold value.

On the other hand, if the maximum point is not detected in the AF evaluation values (NO in step S7), the process proceeds to step S3. In step S3, the fundus camera drives the focus lens 9 by a predetermined amount. The process then returns to step S2, in which the fundus position detection unit 21a again performs pattern recognition. In step S4, the focus detection range determination unit 21b then determines the focus detection range. As a result, the focus detection range can follow the movement of the eye to be examined E, even when the eye to be detected E moves while performing automatic focusing. If pattern recognition or the detection of the maximum point of the AF evaluation values cannot be determined during a predetermined number of cycles, it may be determined as an error.

FIG. 7 illustrates the fundus image displayed on the display monitor 25. Referring to FIG. 7, the relative positions of an optic disc N, large and medium blood vessels V, and a yellow spot Y which are unique to the fundus do not vary greatly regardless of the difference between individuals. Further, the relative positions are mirror-reversed between the left and right eyes.

FIG. 8 illustrates the AF evaluation values when the focus detection range is the region pattern of the large and medium blood vessels V. An AF evaluation value calculation method is a method for easily detecting the high-frequency component in the image. In such a method, luminance signals of a target pixel and eight pixels that are horizontally, vertically, and diagonally adjacent to the target pixel are compared. A value of the greatest difference between the luminance signals of the pixels then becomes the AF evaluation value of the target pixel. An image G1 is an example of a portion of the image in which the large and medium blood vessels V exist in a vertical direction. The luminance signal of each pixel is either “0” or “1”.

When the above-described detection method is applied to the image, the AF evaluation values for each pixel are acquired as illustrated in an image A2. A total sum of the AF evaluation values of the pixels can then be set as the AF evaluation value of the entire image.

The AF evaluation value can be more easily and speedily calculated by comparing the luminance signals of two adjacent pixels . If there is no difference, the AF evaluation value is set to “0”, and if there is a difference, the AF evaluation value is set to “1”. Since the number of pixels to be compared is less than the number in the above-described method, the calculation load is reduced. However, if two pixels which are vertically adjacent to each other in the image G1 are compared, an image G3 is acquired, and edges of the large and medium blood vessels V cannot be detected.

On the other hand, if such a method is applied to an image G4 in which the large and medium blood vessels V exist in a horizontal direction, an image G5 is acquired. A result similar to the image G2 in which the AF evaluation values are calculated using the previously-described method can thus be acquired. In other words, if a detection method which is direction-dependent as the above-described method is selected, the calculation time can be shortened. However, it becomes necessary to appropriately select the target image.

As described above, the differences between the luminance of the adjacent pixels in the images G1 and G4 are mapped as indicated by the images G2 and G5 . If the difference is greater, the difference in the luminance between the adjacent pixels is great, and the total sum is set as the AF evaluation value of the entire image.

The large and medium blood vessels V according to the present exemplary embodiment run in a circular arc shape with the yellow spot Y at proximate center in the fundus Er . The region in which the vessels become thicker exist near the optic disc N so that the edge of the large and medium blood vessels V exist approximately in a direction of plus-minus 45 degrees. High speed automatic focusing can thus be performed with a low load and without lowering the sensitivity of the AF evaluation value by employing a detection method that is selective in such a direction.

According to the present exemplary embodiment, the large and medium blood vessels V in the fundus Er are employed in performing pattern recognition of the fundus image. However, patterns of other regions such as the optic disc N or the yellow spot Y may be stored in the fundus image pattern memory 21d, so that automatic focusing is performed with respect to such regions.

The focus detection range can thus be automatically determined using pattern recognition, and the AF operability can be improved. Further, the focus detection position can follow the movement of the eye to be examined E, so that focus accuracy can be improved.

Furthermore, since the focus state detection unit 21 refers to the luminance value of each pixel when calculating the AF evaluation value, the saturation of the luminance value of the determined focus detection range may be detected. If the luminance value is saturated, the focus state detection unit 21 transmits a signal to the illumination light quantity control unit 24 to adjust the light quantity of the observation light source 1. Automatic focusing can thus be performed with higher accuracy. For example, if the light quantity of the illumination optical system is adjusted when performing contrast detection on the optic disc N in which overexposure is easily generated, a highly accurate fundus image having a great diagnostic value can be acquired.

According to the first exemplary embodiment, pattern recognition is performed on a specific region of the fundus Er. According to a second exemplary embodiment, the examiner selects before starting automatic focusing, the region in the fundus Er for setting the focus detection range. The focus detection range is thus determined based on the selection, and automatic focusing is then performed.

According to the second exemplary embodiment, the fundus image pattern memory 21d includes a plurality of fundus image patterns, such as the region patterns of the optic disc N, the yellow spot Y, and the large and medium blood vessels V. The examiner previously selects the region to be focused according to a case, using a region selection unit such as the cursor on the display monitor 25. This is equivalent to the fundus position detection unit 21a selecting one of the plurality of fundus image patterns . Further, the fundus position detection unit 21a detects a position of the fundus image pattern selected based on the output from the fundus image pickup unit 11 and transfers the result to the focus detection range determining unit 21b. Such a process and the processes to follow are similar to those described according to the first exemplary embodiment.

The examiner may also select a plurality of regions in the fundus instead of one region. In such a case, the AF evaluation value is calculated for each of the plurality of regions, and the sum of the AF evaluation values is set as an overall evaluation value. Accordingly, an image which is averagely focused with respect to the plurality of regions selected by the examiner can be acquired by detecting the maximum value of the overall evaluation value. As a result, the fundus image in which the region desired by the examiner is focused can be photographed, and the examiner can acquire the fundus image of great diagnostic value.

As described above, the fundus image of great diagnostic value can be acquired by performing pattern recognition of the region the examiner particularly desires to focus on in the diagnosis and determining the focus detection range. In other words, an appropriate focus detection range can be determined in the optic disc N, the large and medium blood vessels V, and the yellow spot Y in the fundus image which include a comparatively large amount of high-frequency component. Highly accurate contrast detection can thus be performed.

In particular, highly accurate contrast detection can be performed by detecting the large and medium blood vessels V in which there is little difference between individuals as compared to the optic disc N while there is a great difference between individuals in concavity and convexity of the optic disc N. Further, a running direction of the large and medium blood vessels V can be easily identified. Highly accurate contrast detection with a small calculation load can thus be performed at high speed and low cost by detecting the contrast in a direction perpendicular to the large and medium blood vessels V.

Furthermore, an image of high diagnostic value, appropriate for a lesion which the examiner is focusing on can be acquired by the examiner selecting the focus detection range from the plurality of regions in the fundus.

According to the second exemplary embodiment, the examiner selects the focus detection range before starting automatic focusing. According to the third exemplary embodiment, the examiner selects the focus detection range from specific regions on which pattern recognition has been performed, and automatic focusing is then performed.

According to the first exemplary embodiment, the fundus image pattern memory 21d includes a plurality of fundus image patterns, such as the region patterns of the optic disc N, the yellow spot Y, and the large and medium blood vessels V. This is similar to the second exemplary embodiment. According to the third exemplary embodiment, the positions of the plurality of fundus image patterns are detected with respect to the output from the fundus image pickup unit 11. The result is then transferred to the focus detection range determination unit 21b. Such a process is different from the first and second exemplary embodiments.

Referring to FIG. 9, the focus detection range determination unit 21b according to the third exemplary embodiment includes a focus detection range correction unit 21e and a focus detection range selection unit 21f. The focus detection range display unit 25a included in the display monitor 25 displays to the examiner the plurality of specific regions of the fundus image extracted by the fundus position detection unit 21a. The examiner uses the focus detection range selection unit 21f, i.e., the cursor, and selects from the plurality of specific regions, one region for setting the focus detection range. The specific regions of the fundus image may be displayed to the examiner when a predetermined number of pattern recognition results has been detected, or when the focus lens 9 has moved over the entire movable range.

Further, the examiner can manually correct the position and the size of the focus detection range using the focus detection range correction unit 21e. The examiner can thus acquire the fundus image in which the region desired by the user is correctly focused.

Furthermore, the examiner may also select the plurality of regions instead of one fundus region, similar to the second exemplary embodiment. The process for transferring the selected specific region of the fundus image to the focus detection range determination unit 21b and the processes that follow are similar to the first exemplary embodiment.

According to the second and third exemplary embodiments, the AF evaluation value of one or a plurality of the focus detection ranges selected by the examiner from a plurality of pattern-recognized fundus image regions is calculated. According to a fourth exemplary embodiment, the AF evaluation values are calculated and evaluated for all of the plurality of fundus image region patterns that are pattern-recognized, and automatic focusing is then performed.

According to the fourth exemplary embodiment, the focus state detection unit 21 calculates the AF evaluation values for each of the plurality of specific regions in the fundus image, extracted by the fundus position detection unit 21a. The focus state detection unit 21 then sets the sum of the calculated AF evaluation values as the overall evaluation value. An image which is averagely focused with respect to the plurality of regions selected by the examiner can thus be acquired by detecting the maximum value of the overall evaluation value.

Further, referring to FIG. 10, the focus detection range determination unit 21b according to the fourth exemplary embodiment includes a focus detection range narrowing unit 21g. The focus detection range narrowing unit 21g automatically determines the specific region having the highest AF evaluation value as the focus detection range and transfers the result to the focus state detection unit 21. The process for transferring the selected specific region of the fundus image to the focus detection range determination unit 32b and the processes to follow are similar to the above-described exemplary embodiments. As a result, the focused fundus image can be automatically photographed, so that a fundus camera of high AF operability can be acquired.

In other words, since the focus detection range is automatically determined, the AF operability can be improved.

According to the above-described exemplary embodiments, the position of the specific region in the fundus image is detected only by pattern recognition performed by the fundus position detection unit 21a. According to a fifth exemplary embodiment, pattern recognition of the optic disc N and detection of the right/left eye are combined. The large and medium blood vessels V including a large amount of the specific high-frequency components are then detected, and automatic focusing is performed.

FIG. 11 illustrates an external view of the fundus camera according to the fifth exemplary embodiment. Amount 32 which is movable back and forth and in a horizontal direction as indicated by arrows illustrated in FIG. 11 is disposed on abase 31. A chassis 33 in which the optic system of the fundus camera illustrated in FIG. 1 is included, and the display monitor 25 are disposed on the mount 32. Further, a control stick 35 including a photographing switch is disposed on the mount 32.

The examiner operates with the control stick 35 and adjusts the mount 32 in the horizontal direction to align with the right and left eyes. Aright/left eye detection unit 36 is disposed between the base 31, and the mount 32. The position of the chassis 33 in the horizontal direction is then detected, so that the right/left eye detection unit 36 can detect whether the right eye or the left eye to be examined E is being observed and photographed.

FIG. 12 illustrates a detection method performed by the right/left eye detection unit 36. Referring to FIG. 12, there is a low portion 31a and a high portion 31b on a top surface of the base 31 which form a difference in height. The right/left eye detection unit 36 which is disposed on a bottom surface of the mount 32 is formed of a microswitch. The right/left eye detection unit 36 is in an “off” state when positioned above the low portion of the base 31 and an “on” state when positioned above the high portion of the base 31. More specifically, the left or the right eye to be examined facing the chassis 33 can be detected by forming the low portion 31a on the left side and the high portion 31b on the right side and detecting the on/off state of the right/left eye detection unit 36.

A method for detecting the focus detection range by the right/left eye detection unit 36 performing the right/left eye detection and the fundus position detection unit 21a performing pattern recognition of the optic disc N will be described below. In particular, the method for detecting the large and medium blood vessels V illustrated in FIG. 7 will be described below.

The structure of the fundus Er can be predicted by detecting a specific region in the fundus Er and determining whether the right or left eyes is being observed. The large and medium blood vessels V can then be detected by the right/left eye detection unit 36 detecting the right or left eye and by performing pattern recognition of the optic disc N. The process for transferring the result of detecting the large and medium blood vessels V, to the focus detection range determination unit 21b and the processes to follow are similar to the above-described exemplary embodiments.

According to the present exemplary embodiment, only the optic disc N on which pattern recognition is easily performed is detected. The other regions in the fundus Er are then predicted from the detection result and determined as the focus detection range. The specific region in the fundus Er and the focus detection range may thus be displaced due to the difference between individuals. In such a case, the examiner uses the focus detection range correction unit 21e to manually correct the position and the size of the focus detection range, so that the fundus image in which the region desired by the examiner is correctly focused can be acquired.

As described above, the large and medium blood vessels V or the yellow spot Y is identified and set as the focus detection range by detecting the optic disc N which can be easily pattern-recognized and by detecting the right or left eye. The calculation load and the calculation time are thus reduced, so that high-speed automatic focusing can be realized.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2009-201290 filed Sep. 1, 2009, which is hereby incorporated by reference herein in its entirety.

1. A fundus camera comprising:
a fundus illumination optical system configured to illuminate a fundus of an eye to be examined;
a fundus imaging optical system having a focus lens which is driven to focus on a fundus;
a focus lens driving unit configured to drive the focus lens;
a fundus image pickup unit arranged at a position which is conjugate to a fundus with respect to the fundus imaging optical system;
a display monitor configured to display a fundus image acquired by the fundus image pickup unit;
a focus state detection unit configured to detect an AF evaluation value indicating a level of a focus state based on an output signal from the fundus image pickup unit; and
a lens drive control unit configured to drive the focus lens based on the AF evaluation value detected by the focus state detection unit,
wherein the focus state detection unit includes a fundus position detection unit configured to detect with respect to an output from the fundus image pickup unit a specific region of a fundus image using a region pattern unique to a fundus region, and a focus detection range determination unit configured to determine a focus detection range based on an output from the fundus position detection unit, and calculates the AF evaluation value of the focus detection range determined by the focus detection range determination unit.

2. The fundus camera according to claim 1, wherein the display monitor includes a focus detection range display unit configured to display the focus detection range determined by the focus detection range determination unit, superposing the focus detection range on a fundus image acquired by the fundus image pickup unit.

3. The fundus camera according to claim 1, wherein the region pattern identifies an optic disc of a fundus.

4. The fundus camera according to claim 1, wherein the region pattern identifies large and medium blood vessels of a fundus.

5. The fundus camera according to claim 1, wherein the region pattern identifies a yellow spot of a fundus.

6. The fundus camera according to claim 1, wherein the fundus position detection unit includes a plurality of the region patterns.

7. The fundus camera according to claim 6, wherein the focus detection range determination unit includes a region selection unit configured to cause an examiner to previously select a plurality of the region patterns.

8. The fundus camera according to claim 6, wherein the focus detection range determination unit includes a focus detection range selection unit configured to display a plurality of the focus detection ranges identified by a plurality of the region patterns and causes an examiner to select one or a plurality of focus detection ranges from the displayed plurality of focus detection ranges.

9. The fundus camera according to claim 1, further comprising driving the focus lens based on the AF evaluation value with respect to a plurality of the focus detection ranges identified by a plurality of the region patterns.

10. The fundus camera according to claim 9, wherein the focus detection range determination unit includes a focus detection range narrowing unit configured to determine, based on the AF evaluation value with respect to a plurality of the focus detection ranges, one of the focus detection ranges among a plurality of the focus detection ranges.

11. The fundus camera according to claim 1, further comprising right/left eye detection unit configured to determine whether a left eye or a right eye is observed from a position of a mount on which an optical system is mounted,
wherein the fundus camera detects a position of a large and medium blood vessels or a position of a yellow spot based on a position of an optic disc acquire by the fundus position detection unit and an output from the right/left eye detection unit.

12. The fundus camera according to claim 1, wherein the focus detection range determination unit further includes a focus detection range correction unit configured to correct a position and size of the focus detection range.

13. The fundus camera according to claim 1, further comprising an illumination light quantity control unit configured to adjust an illumination light quantity of an observation light source in the fundus illumination optical system,
wherein the illumination light quantity control unit controls the illumination light quantity based on an output from the focus state detection unit.
